(19) United States
(12) Patent Application Publication
(10) Pub. No.: US 2016/0273163 A1
(43) Pub. Date: Sep. 22, 2016

(54) METHOD FOR PRODUCING ACID WASHED CELLULOSIC FEEDSTOCK FOR ETHANOL PRODUCTION

(71) Applicants: Terry Gong, Moraga, CA (US); Marcus G. Theodore, Salt Lake City, UT (US)

(72) Inventors: Terry Gong, Moraga, CA (US); Marcus G. Theodore, Salt Lake City, UT (US)

(21) Appl. No.: 14/659,365

(22) Filed: Mar. 16, 2015

(51) Int. Cl. D21C 11/00; C07C 29/09; D21C 5/02; C12P 7/10

(52) U.S. Cl. CPC D21C 11/0007; C12P 7/10; C07C 29/09; D21C 11/0021; D21C 5/022; C12P 2203/00

(57) ABSTRACT

A method for producing acid washed wastewater cellulosic feed stock for ethanol production by injecting sulfur dioxide into waters containing cellulosic solids for agglomeration and separation producing acid hydrolyzed conditioned cellulosic materials.

### BACKGROUND OF THE INVENTION

1. Related Applications

This patent application is dependent upon U.S. Provisional Patent Application No. 61/954,123 filed Mar. 17, 2014 entitled “Method for Producing Acid Washed Wastewater Cellulosic Feedstock for Ethanol Production”.

2. Field

This invention pertains to ethanol production methods. More particularly it relates to a method for producing acid washed wastewater cellulosic feedstock for ethanol production by injecting sulfur dioxide into wastewaters or waters containing cellulosic solids and fibers such as dissolved toilet papers found in wastewaters, manure from animal operations such as dairy lagoons, or any type of waters used specifically to separate, capture, and condition solids for ethanol production.

3. Statement of the Art

“Less than 4% of the ethanol is produced synthetically from oil, while the rest is produced by fermentation from bioresources. Ethanol is now produced from two major groups of bioresources: sugar substances and starchy materials. There is a competition between these two feedstocks for fuel ethanol production. While sugar substances were the feedstock for more than 60% of fuel ethanol production at the beginning of the 2000s, its share decreased to 47% by 2006, when grains accounted for 53% of the production (Licht 2006). Ethanol has a potential market as big as the oil market. It can potentially replace the entire fuel market for gasoline. Furthermore, plastics such as polyethylene can be produced from ethanol through ethylene. However, the amounts of sugar substances and grains are limited in the world. They are relatively expensive feedstocks for ethanol production, and ethanol competes with human food for these raw materials. This competition may lead the price of grains and sugar to higher levels in the future. Furthermore, the economy of the ethanol production process from grains depends on the market for its by-product, i.e. distillers' dried grains with solubles (DDGS) as animal food, which may not expand like the ethanol market in the future. In addition to the price, there is concern about damaging forests by increasing farming area as a result of more ethanol production from, for instance, sugar cane in Brazil. Lignocellulosic materials are renewable, largely unused, and abundantly available sources of raw materials for the production of fuel ethanol. Lignocellulosic materials can be obtained at low cost from a variety of resources, e.g. forest residues, municipal solid waste, waste paper, animal operations such as from dairy lagoons and crop residue resources (Wyman 1996). These materials contain sugars polymerized in form of cellulose and hemicellulose, which can be liberated by hydrolysis and subsequently fermented to ethanol by microorganisms (Millati et al. 2002; Palmqvist and Hahn-Hägerdal 2000),” See PEER-REVIEWED REVIEW ARTICLE, ncsu.edu/bioresources, Taherzadeh and Karimi (2007). “Bioethanol review,”BioResources 2(3), 472-499.

In view of the need for renewable cellulosic feedstocks, a number of researchers have found that municipal wastewater solids may be used as feedstocks for the recovery of simple sugars; see Abstract, “Enzymatic hydrolysis of cellulosic municipal wastewater treatment process residuals as feedstocks for the recovery of simple sugars”, by Pascale Chamgne, Caljian Li, Bioresource Technology, Volume 100, issue 23, December 2009, Pages 5700-5706; and recovery of cellulose, “Recovery of biomass cellulose from waste sewage sludge”; Abstract, Journal of Material Cycles and Waste Management, April 2002, Volume 4, Issue 1, pp 46-50, and “Waste autoclave”; http://en.wikipedia.org/wild/Waste_autoclave.

The present method provides a sustainable acid washed cellulosic feedstock suitable for conversion to cellulosic ethanol.

### SUMMARY OF THE INVENTION

The present invention comprises injecting sufficient SO2 to transform waters containing cellulosic fibrous materials to lower the pH and provide free SO₂, sulfites and bisulfites to chemically dewater and separate the solids from the wastewater to provide recovered acid treated cellulosic feedstock particularly suited for acid hydrolysis. Wastewater, as used herein, is principally directed to domestic sewage from dwellings, business buildings, institutions, containing ground water, surface water, and/or storm water, and can include wastewater, having already undergone primary and secondary treatment according to conventional treatment processes. However, if the method is employed as a primary treatment process, additional sources of fibrous material feedstock can be added to increase the overall content and volume of the cellulosic fiber produced and captured. Large cellulosic supplemental sources of fiber may be derived from feedlots, wherein the sulfurous acid not only hydrolyzes the materials, but suppresses odors from their lagoons.

The method comprises chemically treating waters containing cellulosic solids, such as wastewaters, with sulfur dioxide to condition the water and separate the solids. The sulfur dioxide forms sulfurous acid, (H₂SO₃), dissociating to produce H⁺, bi-sulfite (HSO₃⁻), sulfite (SO₃⁻), and free SO₂ species in solution, all hereinafter all referred to as sulfurous acid. The suspended solids are conditioned with sufficient SO₂ to ensure they will chemically dewater when allowed to drain, forming a fairly dry acid washed solid. (7% water) with a pH of 4.6, a BTU content of 6,932 BTU/lb approximating wood chips or shavings.

This dry, slightly acidic acid washed solid provides an ideal feedstock rich in cellulosic material particularly suitable for chemical hydrolysis, using concentrated-acid hydrolysis or dilute-acid hydrolysis. Concentrated sulfuric or hydrochloric acids give high glucose yields from lignocellulosic materials, which are suitable for fermentation into ethanol. Dilute-acid hydrolysis may also be employed, but may form undesirable by-products from the degradation of the sugars adversely impacting the fermentation process, resulting in lower yields. By using the chemically dried feedstock, less concentrated acid is required due to the absence of dilution.

For fermentation applications, the chemical feedstock may be employed in a slurry, which is pH adjusted to that required to promote microbial degradation and production of ethanol. Usually a pH of greater than 4 will insure that the biocidal action of the bisulfites will not harm the microbes in the fermentation vats. Preferably, an organic chemical, such as lime, is used for raising the pH, resulting in a land-applicable process effluent.

The present invention may be embodied in other specific forms without departing from its structures, methods, or other essential characteristics as broadly described herein and claimed hereinafter. The described embodiments are to be considered in all respects only as illustrative, and not restrictive. The scope of the invention is, therefore, indicated by the appended claims, rather than by the foregoing description. All changes that come within the meaning and range of equivalency of the claims are to be embraced within their scope.

1. A method for producing acid washed cellulosic feedstock from waters containing paper and/or suspended solids comprising:
a. injecting sufficient sulfurous acid into the waters containing paper and suspended solids to agglomerate the paper and/or water solids, and
b. separating the paper and/or water solids and allowing them to chemically dry to form an acidic biosolid, high in cellulosic content for use as a feedstock for acid hydrolysis and/or fermentation of the feedstock to produce ethanol.

2. A method for producing acid washed cellulosic feedstock according to claim 1, wherein the waters are wastewater with a pH between 1.5 and 4.5 after the addition of sulfurous acid.

3. A method for producing acid washed cellulosic feedstock according to claim 2, including adding additional cellulosic fibers from waters containing paper and/or suspended cellulosic solids.

4. A method for producing acid washed cellulosic feedstock, according to claim 1, wherein the cellulosic feedstock is derived from feedlots, and the sulfurous acid suppresses odors while acid hydrolyzing the cellulosic material.

5. A method for producing acid washed cellulosic feedstock according to claim 1, wherein the amount of sulfurous acid is dependent upon the buffering alkalinity and oxygen levels in the water.

6. (canceled)

7. A method for producing acid washed cellulosic feedstock according to claim 1, wherein the sulfurous acid waters containing paper and/or suspended solids forms a slurry that is pH adjusted for microbial fermentation into ethanol.

8. A method for producing acid washed cellulosic feedstock according to claim 7, wherein the pH of the slurry is raised above 4.

9. A method for producing acid washed cellulosic feedstock according to claim 7, wherein lime is used to raise the pH of the slurry.
